(19) United States
(12) Patent Application Publication
(10) Pub. No.: US 2011/0077517 A1
(43) Pub. Date: Mar. 31, 2011

(54) Ultrasonic diagnostic apparatus

(75) Inventor: Yoshiaki Satou, Kanagawa (JP)

(73) Assignee: FUJIFILM Corporation, Tokyo (JP)

(21) Appl. No.: 12/923,361

(22) Filed: Sep. 16, 2010

(30) Foreign Application Priority Data
Sep. 30, 2009 (JP) 2009-227547

(51) Int. Cl. A61B 8/14

(52) U.S. Cl. 600/443

(57) ABSTRACT

An ultrasonic probe of an ultrasonic diagnostic apparatus includes an ultrasonic transducer array having a plurality of channels. Each channel contains a pair of a first ultrasonic transducer and a second ultrasonic transducer. The first ultrasonic transducer transmits and receives an ultrasonic wave of a fundamental frequency, and outputs a first reception signal. The second ultrasonic transducer receives a harmonic wave, and outputs a second reception signal. In a normal mode, composite reception signals in which the first reception signals and the second reception signals are added on a pair basis are inputted to a reception circuit. Due to a resonant circuit, a fundamental component of the composite reception signals is inputted to the reception circuit. In a THI mode, only a second harmonic component of the second reception signal is transmitted to the reception circuit due to the resonant circuit.

### BACKGROUND OF THE INVENTION

1. Field of the Invention

The present invention relates to an ultrasonic diagnostic apparatus, which applies an ultrasonic wave to a human body part and images the inside of the body part based on an echo of the ultrasonic wave.

2. Description Related to the Prior Art

An ultrasonic diagnostic apparatus is used for examination of a fetus in utero and various internal body parts including lacteal gland, thyroid gland, and the like, because of the advantage of noninvasively imaging the inside of tissue of the body part in real time. In a conventional ultrasonic diagnostic apparatus, ultrasonic transducers apply an ultrasonic wave of a predetermined frequency to the human body part to be imaged. Then, the same ultrasonic transducers receive an echo of the ultrasonic wave, and output reception signals based on the echo. The ultrasonic diagnostic apparatus images a cross section of the body part based on the reception signals.

Each ultrasonic transducer has a piezoelectric element that is made of a piezoelectric material such as lead zirconate titanate (PZT), for example, formed into a predetermined shape. Upon application of a pulse voltage to front and bottom surfaces of the piezoelectric element, the piezoelectric element repeats expansion and contraction, and emits the ultrasonic wave. When the echo from the internal body part is incident upon the ultrasonic transducer, on the other hand, the piezoelectric element expands or contracts. The expansion or contraction brings about electric potential difference between the front and bottom surfaces of the piezoelectric element, and produces the reception signal. Since the resonant frequency of the piezoelectric element is determined by the size and shape of the piezoelectric element, the ultrasonic transducer mainly emits the ultrasonic wave of the resonant frequency (hereinafter called fundamental frequency), and outputs the reception signal in which a fundamental frequency component (hereinafter called fundamental component) of the incident echo is mainly reflected.

Furthermore, it is known that the echo contains components the frequencies of which are other than the fundamental frequency. This is because dispersion of the ultrasonic wave by a living body is a nonlinear phenomenon, and these components having the frequencies other than the fundamental frequency reflect detailed tissue structure of the internal body part. Thus, a method called harmonic imaging is recently used, in which the component (hereinafter called harmonic component) the frequency of which is an integer multiple of the fundamental frequency is used for production of an ultrasonic image. The harmonic imaging can reduce adverse effects of multiple reflection and side lobe. As a result, the ultrasonic image produced with use of the harmonic component has better lateral, resolution and contrast resolution than those of the ultrasonic image produced only from the fundamental component, and hence the sharper ultrasonic image is obtained (refer to Japanese Patent No. 4192598 and Japanese Patent Laid-Open Publication No. 11-276478).

Conventionally, the ultrasonic diagnostic apparatus was large stationary equipment set up in a large hospital. However, the portable ultrasonic diagnostic apparatus, which can be set up in a medical clinic or carried about bedsides of a hospital ward for use, is widely available in recent years. In such a portable ultrasonic diagnostic apparatus, it is desired to reduce power consumption as much as possible, considering that the portable ultrasonic diagnostic apparatus is driven with electric power supply only from an internal battery. However, if the ultrasonic transducers for transmitting and receiving the ultrasonic wave are driven at a low voltage, the echo itself from the internal body part is weakened. This causes a shortage of sensitivity and degradation in image quality of the ultrasonic image. Especially, since the harmonic component of the echo is conspicuously reduced, it becomes difficult to observe the detailed tissue structure and make a correct diagnosis.

The ultrasonic diagnostic apparatus is constituted of an ultrasonic probe and an ultrasonic observing device (processor device) that processes the reception signals obtained by the ultrasonic probe and displays the ultrasonic image. A cable for connecting the ultrasonic probe to the ultrasonic observing device sometimes interferes with operation of the ultrasonic probe. The portable ultrasonic diagnostic apparatus, in particular, is small in size and light in weight. Thus, if the cable is thick or rigid, the ultrasonic observing device moves together with movement of the ultrasonic probe, and causes interference with the diagnosis. For this reason, it is desired to reduce the diameter of the cable or eliminate the cable by using wireless communication between the ultrasonic probe and the ultrasonic observing device.

### SUMMARY OF THE INVENTION

A main object of the present invention is to provide an ultrasonic diagnostic apparatus that can sensitively receive a harmonic component at low power.

Another object of the present invention is to provide the ultrasonic diagnostic apparatus having an ease-to-operate ultrasonic probe by reducing the diameter of a cable between the ultrasonic probe and an ultrasonic observing device.

To achieve the above and other objects, an ultrasonic diagnostic apparatus according to the present invention includes an ultrasonic probe and an ultrasonic observing device. The ultrasonic probe includes an ultrasonic transducer array, a reception circuit, a detector, a parallel-to-serial converter, a switching device, and a controller. The ultrasonic transducer array has a plurality of channels arranged in a line. Each of the channels has a pair of a first ultrasonic transducer for transmitting and receiving an ultrasonic wave of a fundamental frequency and a second ultrasonic transducer for receiving a harmonic wave having a frequency of an integer multiple of the fundamental frequency. The reception circuit amplifies a first reception signal from the first ultrasonic transducer and a second reception signal from the second ultrasonic transducer, and applies analog-to-digital conversion to the first and second reception signals. The detector detects an output signal from the reception circuit with use of a reference signal with a predetermined angular frequency. The parallel-to-serial converter converts an output signal from the detector into a serial signal. The switching device switches between a first mode and a second mode. In the first mode, the first reception signal from the first ultrasonic transducer and the second reception signal from the second ultrasonic transducer are added on a pair basis, and inputted to the reception circuit. In the second mode, only the second reception signal from the second ultrasonic transducer is inputted to the reception circuit. The controller changes the angular frequency of the reference signal in accordance with a state of the switching device. The ultrasonic observing device produces an ultrasonic image from the serial signal transmitted from the ultrasonic probe.

The ultrasonic diagnostic apparatus may further include a resonant circuit having a variable resonant frequency disposed between the second ultrasonic transducer and the reception circuit. The controller determines the angular frequency in accordance with the resonant frequency.

The controller may set the resonant frequency at the fundamental frequency in the first mode, and set the resonant frequency at a frequency of the harmonic wave in the second mode.

In the first mode, the resonant frequency may be changed in accordance with reception timing of the ultrasonic wave.

The resonant circuit may include an inductor and a variable capacitance capacitor connected in parallel. The resonant frequency is adjusted by varying a capacitance of the variable capacitance capacitor. The variable capacitance capacitor may be a varicap.

It is preferable that the first ultrasonic transducer has a piezoelectric element made of an inorganic material, and the second ultrasonic transducer has a piezoelectric element made of an organic material.

The pair of the first ultrasonic transducer and the second ultrasonic transducer may be stacked.

It is preferable that the ultrasonic probe and the ultrasonic observing device are portable.

The ultrasonic diagnostic apparatus may further include a cable for transmitting the serial signal from the ultrasonic probe to the ultrasonic observing device. The cable adheres to one of standards of USB3.0, sATAgen2, and 10 GbaseT.

The serial signal may be transmitted by wireless communication from the ultrasonic probe to the ultrasonic observing device.

According to the present invention, the ultrasonic probe can sensitively receive the harmonic component, even if driven at a low voltage. The present invention can reduce the diameter of the cable between the ultrasonic probe and the ultrasonic observing device, and facilitates handling of the ultrasonic probe.

### BRIEF DESCRIPTION OF THE DRAWINGS

For more complete understanding of the present invention, and the advantage thereof, reference is now made to the following descriptions taken in conjunction with the accompanying drawings, in which:

FIG. 1 is a perspective view of a portable ultrasonic diagnostic apparatus;

FIG. 2 is a block diagram of the ultrasonic diagnostic apparatus;

FIG. 3 is a circuit diagram in a normal mode;

FIG. 4 is a circuit diagram in a tissue harmonic imaging (THI) mode;

FIG. 5 is a timing chart in an operation state of the ultrasonic diagnostic apparatus;

FIG. 6 is a graph showing the sensitivity of an ultrasonic transducer; and

FIG. 7 is a timing chart in another operation state of the ultrasonic diagnostic apparatus.

### DESCRIPTION OF THE PREFERRED EMBODIMENTS

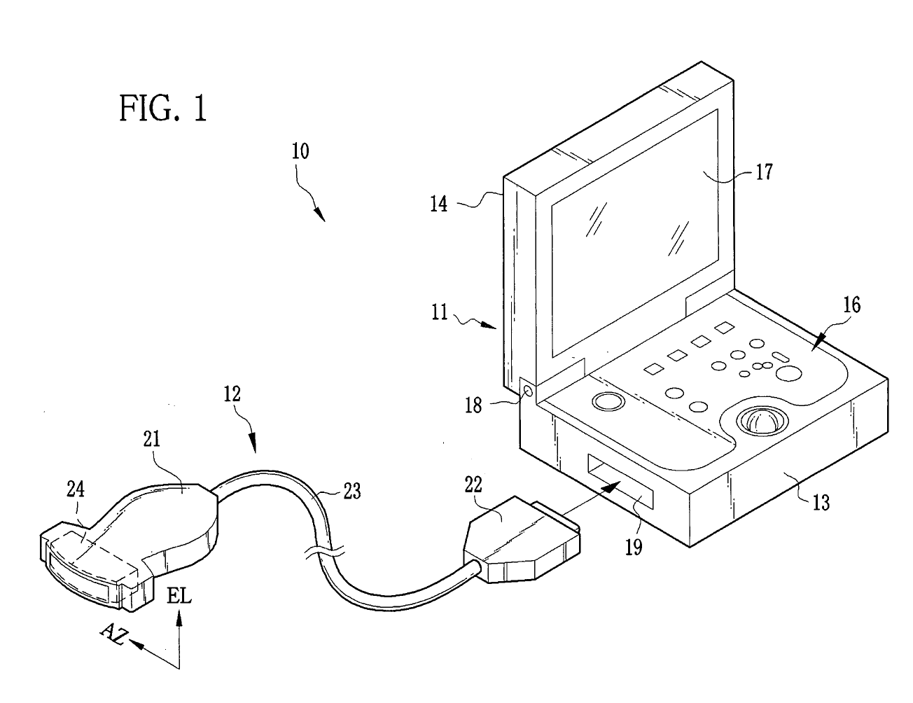

As shown in FIG. 1, a portable ultrasonic diagnostic apparatus 10 is constituted of an ultrasonic observing device (processor device) 11 and an ultrasonic probe 12. The ultrasonic observing device 11 is composed of a main body 13 and a cover 14. On a top surface of the main body 13, an operation unit 16 having a plurality of buttons and a trackball for inputting various operation commands is provided. Inside of the cover 14, there is provided a monitor 17 (for example, liquid crystal display) for displaying an ultrasonic image and various operation screens.

The cover 14 is hinged on the main body 13 with a hinge 18, and is rotatable between an open position in which the operation unit 16 and the monitor 17 are exposed, and a closed position in which the top surface of the main body 13 is faced to an inner surface of the cover 14 to cover both of the operation unit 16 and the monitor 17 for protection. A grip (not illustrated) is attached to a side surface of the main body 13 to make the ultrasonic observing device 11 convenient to carry about in a state of closing the main body 13 and the cover 14. In the other opposite side surface of the main body 13, there is provided a probe connection portion 19 to which the ultrasonic probe 12 is detachably connected.

The ultrasonic probe 12 is constituted of a scan head 21, which a doctor holds and presses against a human body part to be imaged, a connector 22 connected to the probe connection portion 19, and a cable 23 for connecting the scan head 21 to the connector 22. The scan head 21 contains an ultrasonic transducer array 24 at its distal end. In the ultrasonic transducer array 24, ultrasonic transducers for composing a plurality of channels are aligned in an azimuth (AZ) direction.

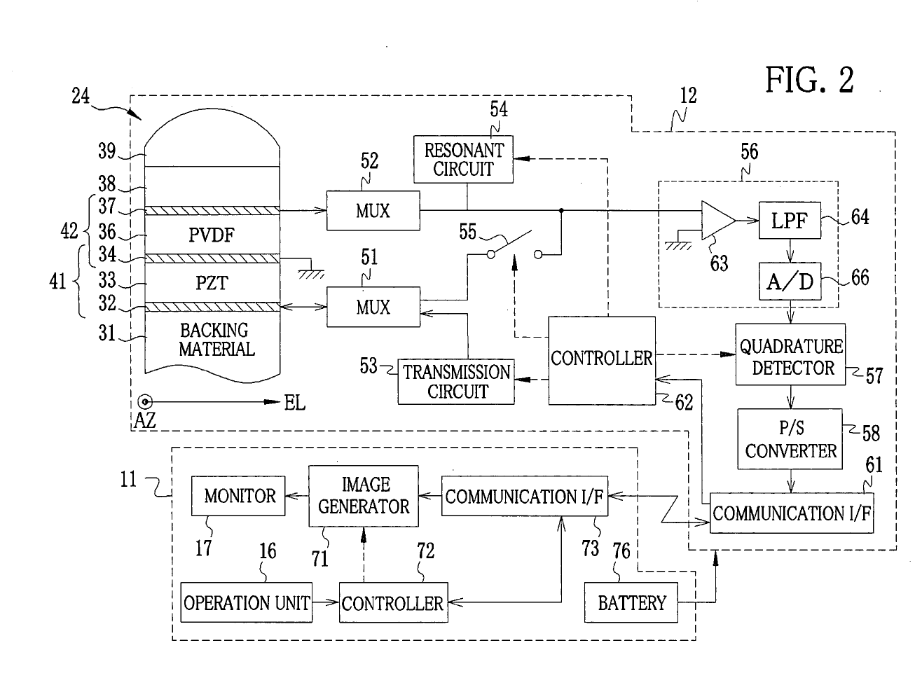

Viewing a cross section of the ultrasonic transducer array 24 in an elevation (EL) direction, as shown in FIG. 2, the ultrasonic transducer array 24 has such structure that a backing material 31, a first electrode 32, a first piezoelectric element 33, a common electrode 34, a second piezoelectric element 36, a second electrode 37, an acoustic impedance matching layer 38, and an acoustic lens 39 are stacked in this order on a plate-shaped mount support (not illustrated) made of a glass-epoxy resin or the like. The first electrode 32, the first piezoelectric element 33, and the common electrode 34 compose a first ultrasonic transducer 41. The common electrode 34, the second piezoelectric element 36, and the second electrode 37 compose a second ultrasonic transducer 42. Thus, in the ultrasonic transducer array 24, the single first ultrasonic transducer 41 and the single second ultrasonic transducer 42 are stacked in a single channel. Each of the first and second ultrasonic transducers 41 and 42 has the shape of a block long in the EL direction. A lot of pairs of the stacked first and second ultrasonic transducers 41 and 42 are aligned in the AZ direction at regular intervals via a filling material therebetween.

The backing material 31 is made of an epoxy resin, a silicone resin, or the like, and absorbs ultrasonic wave that is emitted from the first ultrasonic transducer 41 in the direction of the mount support. The backing material 31 is in a gentle dome shape at a top surface, and has a convex cross-section in the AZ direction, which is orthogonal to the EL direction.

The first electrode 32 is so disposed as to sandwich the first piezoelectric element 33 together with the common electrode 34. To the first electrode 32, a drive pulse (Tx) is inputted to drive the first piezoelectric element 33. In addition, upon reception of echo from the internal body part by the first piezoelectric element 33, a reception signal is obtained through the first electrode 32.

The first piezoelectric element 33 is made of an inorganic material such as lead zirconate titanate (PZT), for example. Each first piezoelectric element 33 has the shape of a block long in the EL direction. Upon input of the drive pulse to the first electrode 32, the first piezoelectric element 33 corresponding to the first electrode 32 expands and contracts in response to the drive pulse, and generates the ultrasonic wave of a frequency (fundamental frequency) f₁, which is determined by the shape of the first piezoelectric element 33. Upon reception of the echo from the internal body part, on the other hand, the first piezoelectric element 33 generates electric potential difference between the first electrode 32 and the common electrode 34 in accordance with the echo. The electric potential difference produces a first reception signal, which is obtained through the first electrode 32. Since the resonant frequency of the first piezoelectric element 33 depends on the shape of the first piezoelectric element 33, the first reception signal obtained from the first piezoelectric element 33 is sensitive to the fundamental frequency f₁ and the vicinity thereof. The first ultrasonic transducer 41 is used for both of transmission and reception.

The common electrode 34 is disposed between the first piezoelectric element 33 and the second piezoelectric element 36, and establishes a ground connection on a package of the scan head 21. The common electrode 34 also functions as an acoustic impedance matching layer that relieves the difference in acoustic impedance between the first piezoelectric element 33 and the second piezoelectric element 36.

The second piezoelectric element 36 is made of an organic material such as polyvinylidene fluoride (PVDF), for example. Each second piezoelectric element 36, as with the first piezoelectric element 33, has the shape of a block long in the EL direction. Although the second piezoelectric element does not clearly have a resonance characteristic because of being made of the organic material, the thickness of the second piezoelectric element 36 is so designed as to mainly resonate with the second harmonic wave (frequency of 2 f₁) of the echo. Upon reception of the echo, the second piezoelectric element 36 generates the electric potential difference between the common electrode 34 and the second electrode 37 in accordance with the echo. From the electric potential difference, a signal is produced in which wide frequency components including a second harmonic component are reflected. At the same time, the second piezoelectric element 36 functions as the acoustic impedance matching layer, and relieves the difference in the acoustic impedance from an ambient structure. The second piezoelectric element 36 does not generate the ultrasonic wave, and thus is used only for the reception.

The second electrode 37 is so disposed as to sandwich the second piezoelectric element 36 together with the common electrode 34. As described above, the electric potential difference generated between the common electrode 34 and the second electrode 37 by the second piezoelectric element 36 in response to the reception of the echo yields a second reception signal outputted from the second electrode 37.

The acoustic impedance matching layer 38 relieves the difference in the acoustic impedance between the ultrasonic transducer array 24 and a human body. The acoustic lens 39 is made of a silicone resin or the like, and has a convex cross section in the EL direction. Therefore, the acoustic lens 39 focuses the ultrasonic wave generated from the first ultrasonic transducer 41 on the internal body part to be imaged in the EL direction.

The ultrasonic probe 12 is provided with multiplexers (MUXs) 51 and 52, a transmission circuit 53, a resonant circuit 54, a reception circuit 56, a quadrature detector 57, a parallel-to-serial converter 58, a communication interface 61, and a controller 62, in addition to the ultrasonic transducer array 24 having above structure.

The MUX 51 successively connects the transmission circuit 53 to the single first ultrasonic transducer 41 selected out of the plurality of first ultrasonic transducers 41. Upon reception of the echo, the MUX 51 successively connects one of the plurality of first ultrasonic transducers 41 to the reception circuit 56 through a mode change switch (hereinafter simply called switch) 55. The MUX 52 also connects the single second ultrasonic transducer 42 selected out of the plurality of the second ultrasonic transducers 42 to the reception circuit 56. The first and second ultrasonic transducers 41 and 42 are grouped by the MUXs 51 and 52, and driven from group to group. In the adjoining groups, the ultrasonic transducers 41, 42 are partly overlapped.

The transmission circuit 53 inputs the drive pulse to the first ultrasonic transducer 41 connected through the MUX 51. The transmission circuit 53 successively inputs the drive pulse to each of the first ultrasonic transducers 41 belonging to the same group with predetermined time delay. Thus, the ultrasonic transducer array 24 scans the internal body part with an ultrasonic beam, which is focused at a predetermined depth in the AZ direction.

The resonant circuit 54 is connected in parallel with the second ultrasonic transducer 42 in the vicinity of the second ultrasonic transducer 42. The resonant frequency of the resonant circuit 54 is variable, so that the resonant circuit 54 can adjust the frequency of the second reception signal inputted from the second ultrasonic transducer 42 to the reception circuit 56. If the switch 55 is turned off, the second reception signal is inputted by itself from the second ultrasonic transducer 42 to the reception circuit 56. At this time, adjusting the resonant frequency of the resonant circuit 54 can select the frequency of the reception signal (the second reception signal) inputted to the reception circuit 56. If the switch 55 is turned on, on the other hand, both of the first reception signal from the first ultrasonic transducer 41 and the second reception signal from the second ultrasonic transducer 42 travel the same signal output line, and are inputted to the reception circuit 56 as a composite reception signal added from pair to pair. At this time, the resonant circuit 54 acts only on a second reception signal component out of the composite reception signal of each pair. Thus, if the switch 55 is turned on, the composite reception signal, into which the first reception signal and the second reception signal having the frequency selected by the resonant circuit 54 are added from pair to pair, is inputted to the reception circuit 56.

The reception circuit 56 includes a plurality of sets of amplifiers 63, low-pass filters (LPFs) 64, and analog-to-digital converters (A/Ds) 66. To the reception circuit 56, depending on a state of the switch 55 as described above, the analog composite reception signal into which the first reception signal and the second reception signal are added is inputted if the switch 55 is turned on, and the analog second reception signal obtained from the second ultrasonic transducer 42 is inputted if the switch 55 is turned off. In the reception circuit 56, the amplifier 63 amplifies the inputted reception signal, and the LPF 64 removes noise of high frequencies. Then, the A/D 66 converts the analog reception signal into the digital reception signal, which is then inputted to the quadrature detector 57. The number of sets of the amplifiers 63, the LPFs 64, and the A/Ds 66 corresponds with the number of the first and second ultrasonic transducers 41 and 42 belonging to the single group, which are selected by the MUXs 51 and 52 on an occasion of reception of the echo. Thus, the reception circuit 56 simultaneously applies above processing to the plurality of reception signals inputted on the single occasion, and inputs to the quadrature detector 57 the processed reception signals in parallel with one another.

The quadrature detector 57 applies quadrature detection processing to each of the reception signals inputted from the reception circuit 56 to produce an I-phase signal and a Q-phase signal, and sampling processing at a predetermined sampling frequency to produce a complex baseband signal. The quadrature detector 57, as described later on, carries out the quadrature detection processing with use of a reference signal, which depends on the resonant frequency of the resonant circuit 54. The quadrature detector 57, as described above, simultaneously applies the quadrature detection processing to the plurality of reception signals inputted from the reception circuit 56 at the same time to produce the complex base band signals, and inputs the complex base band signals to the parallel-to-serial converter 58.

The parallel-to-serial converter 58 converts the plurality of complex baseband signals inputted in parallel from the quadrature detector 57 into a serial reception signal. The serial reception signal is transferred to the ultrasonic diagnostic apparatus 11 with a predetermined protocol through a communication interface 61, which includes the connector 22, the cable 23, and the like. Information and the like inputted from the operation unit 16 is inputted to the controller 62 of the ultrasonic probe 12 through the communication interface 61.

The controller 62 is connected to each part inside the ultrasonic probe 12, to overall control the ultrasonic probe 12. For example, the controller 62 controls the transmission circuit 53 so that the predetermined ultrasonic beam is emitted from the ultrasonic transducer array 24, as described above. The controller 62 switches operation modes of the ultrasonic diagnostic apparatus 10 by switching a turn on or off of the switch 55 in response to input from the operation unit 16. In accordance with a state of the switch 55 and the like, the controller 62 adjusts the resonant frequency of the resonant circuit 54, and controls the quadrature detector 57 to carry out the quadrature detection processing with use of the reference signal corresponding to the adjusted resonant frequency of the resonant circuit 54.

The ultrasonic observing device 11 is provided with an image generator 71, a controller 72, a battery 76, and the like. The image generator 71 generates the ultrasonic image from the reception signal transmitted from the ultrasonic probe 12. At this time, the image generator 71 first converts the reception signal obtained through the communication interface 73 back into original parallel data. The image generator 71 applies reception focusing processing to the parallel data by phase addition, and produces acoustic ray data along predetermined scanning lines. Then, the image generator 71 produces a B-mode image or an M-mode image of the ultrasonic image from the acoustic ray data of a single frame in accordance with setting, and displays the B-mode or M-mode image on the monitor 17.

The controller 72 overall controls each part of the ultrasonic observing device 11 in accordance with input from the operation unit 16, and inputs a control signal to the controller 62 of the ultrasonic probe 12 through the communication interface 73 to control operation of the ultrasonic probe 12.

The battery 76 supplies electric power to each part of the ultrasonic observing device 11, and supplies electric power to each part of the ultrasonic probe 12 through the probe connection portion 19, the connector 22, the cable 23, and the like (refer to FIG. 1).

The ultrasonic diagnostic apparatus 10 having above structure is switchable by turning on or off of the switch 55 between two modes, that is, a normal mode in which the ultrasonic image is produced from a fundamental component of the echo, and a tissue harmonic imaging (THI) mode in which the ultrasonic image is produced from a harmonic component of the echo. In either of the two operation modes, upon input of the drive pulses from the transmission circuit 53 to the first ultrasonic transducers 41, the ultrasonic beam is applied from the ultrasonic transducer array 24 to the body part to be imaged. In either of the normal mode and the THI mode, the ultrasonic diagnostic apparatus 10 drives the ultrasonic transducer array 24 at a low voltage, in order to reduce power consumption of the battery 76.

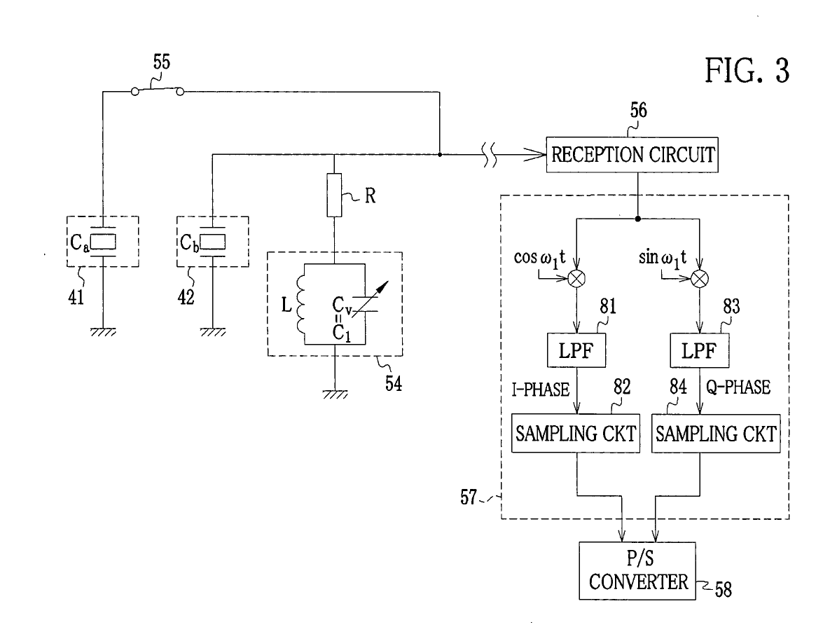

When the ultrasonic diagnostic apparatus 10 is in the normal mode, as shown in FIG. 3, the switch 55 is turned on, and a signal output line of the first ultrasonic transducer 41 is connected to a signal output line of the second ultrasonic transducer 42. Thus, in the normal mode, the composite reception signal, into which the first reception signal from the first ultrasonic transducer 41 and the second reception signal from the second ultrasonic transducer 42 are added, is inputted to the reception circuit 56.

The first ultrasonic transducer 41 is regarded as a capacitor having a capacitance of Cₐ, and the second ultrasonic transducer 42 is regarded as a capacitor having a capacitance of C_{b}. The resonant circuit 54 is composed of an inductor (hereinafter called inductor L) having an inductance of L and a capacitor (hereinafter called variable capacitance capacitor Cᵥ,) having a capacitance of Cᵥ that are connected in parallel. The resonant circuit 54 is connected to the signal output line via a damping resistance R. As the variable capacitance capacitor Cᵥ, a variable capacitance diode (so-called varicap) is used, in which the thickness of a depletion layer is actively variable in accordance with the magnitude of an applied direct-current voltage.

In the normal mode, the controller 62 of the ultrasonic probe 12 sets the variable capacitance capacitor Cᵥ at C₁ satisfying

$${f}_{1} = \frac{1}{2 π \sqrt{L \times {C}_{1}}} ,$$

so that the resonant frequency of the resonant circuit 54 becomes the fundamental frequency f₁. The resonant circuit 54 functions as a circuit that has almost infinite impedance to a signal of the fundamental frequency f₁, and has lower impedance than that of the reception circuit 56 to signals other than the fundamental frequency f₁. Therefore, components of the second reception signal having frequencies other than the fundamental frequency f₁ are absorbed to ground through the resonant circuit 54. On the other hand, a component of the second reception signal having the fundamental frequency f₁ is transmitted to the reception circuit 56 through the signal output line. As a result, the composite reception signal inputted to the reception circuit 56 is an addition of the first reception signal, which is outputted from the first ultrasonic transducer 41 and almost only has the component of the fundamental frequency f₁, and the fundamental component of the second reception signal outputted from the second ultrasonic transducer 42, on a pair basis. The controller 62 also set an angular frequency ω of the reference signal used in the quadrature detector 57 at ω₁ satisfying

$${ω}_{1} = \frac{{f}_{1}}{2 π} .$$

The quadrature detector 57 divides each reception signal outputted from the reception circuit 56 in two. One of the divided reception signal is multiplied by a reference signal cos ω₁t, and is passed through a low-pass filter (LPF) 81 to produce the I-phase signal. Then, since a sampling circuit 82 applies sampling processing to the I-phase signal with a predetermined sampling frequency, the baseband I-phase reception signal is inputted to the parallel-to-serial converter 58. The other one of the divided reception signal is multiplied by a reference signal sin ω₁t, and is passed through a low-pass filter (LPF) 83 to produce the Q-phase signal. Then, a sampling circuit 84 applies sampling processing to the Q-phase signal, as with the sampling circuit 82, so that the baseband Q-phase reception signal is inputted to the parallel-to-serial converter 58.

The parallel-to-serial converter 58 coverts the reception signals processed as described above into serial data, and transfers the serial data to the ultrasonic observing device 11. The ultrasonic observing device 11 produces the ultrasonic image from the reception signals obtained as described above, and display the ultrasonic image on the monitor 17. Thus, the tomographic image displayed on the monitor 17 in the normal mode visualizes the internal body part with the fundamental component of the echo.

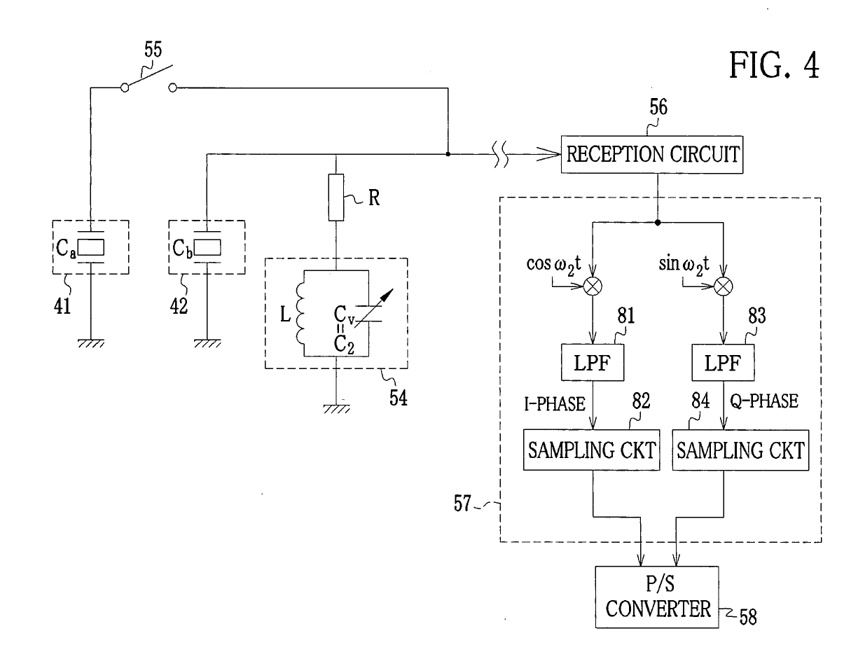

When the ultrasonic diagnostic apparatus 10 is in the THI mode, as shown in FIG. 4, the switch 55 is turned off, and the signal output line connected to the first ultrasonic transducer 41 is cut off from the reception circuit 56. Thus, only the second reception signal from the second ultrasonic transducer 42 is inputted to the reception circuit 56.

In the THI mode, the controller 62 of the ultrasonic probe 12 sets the variable capacitance capacitor Cᵥ at C₂ satisfying

$$2 {f}_{1} = \frac{1}{2 π \sqrt{L \times {C}_{2}}} ,$$

so that the resonant frequency of the resonant circuit 54 becomes the second harmonic frequency f₂. Thus, the resonant circuit 54 functions as a circuit that has almost infinite impedance to a signal of the frequency 2 f₁, and has lower impedance than that of the reception circuit 56 to signals having frequencies of other than the frequency 2 f₁. The reception signal inputted to the reception circuit 56 contains only the second harmonic component of the second reception signal outputted from the second ultrasonic transducer 42. Thus, the ultrasonic diagnostic apparatus 10 is sensitive to the second harmonic component, even if driven at the low voltage. The controller 62 also set an angular frequency ω of the reference signal used in the quadrature detector 57 at ω₂ satisfying

$${ω}_{2} = \frac{2 {f}_{1}}{2 π} .$$

As in the case of the normal mode, the quadrature detector 57 divides each reception signal outputted from the reception circuit 56 in two. The divided reception signals are multiplied by reference signals (cos ω₂t and sin ω₂t) of the angular frequency ω₂ determined as above, and are passed through the LPFs 81 and to produce the I-phase signal and the Q-phase signal, respectively. Then, since the sampling circuits 82 and 84 apply the sampling processing to the I-phase signal and the Q-phase signal, respectively, the baseband I-phase reception signal and the baseband Q-phase reception signal are inputted to the parallel-to-serial converter 58.

After that, as in the case of the normal mode, the ultrasonic observing device 11 produces the ultrasonic image, and displays the ultrasonic image on the monitor 17. The ultrasonic image generated from the second harmonic component is displayed in the THI mode, though the ultrasonic image generated from the fundamental component is displayed in the normal mode. Consequently, although the ultrasonic transducer array 24 is driven at the low voltage in both of the normal mode and the THI mode, the ultrasonic image obtained in the THI mode has higher definition than that of the ultrasonic image obtained in the normal mode.

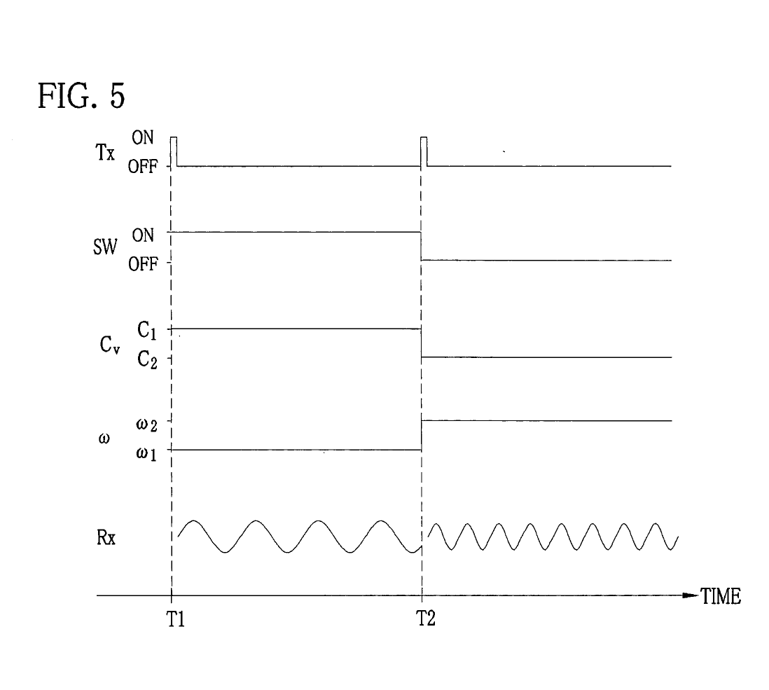

The ultrasonic diagnostic apparatus 10 can be switched between the normal mode and the THI mode at almost arbitrary timing with the operation from the operation unit 16. Now, a case is considered, as shown in FIG. 5, where after the drive pulse Tx for transmitting the ultrasonic wave from the ultrasonic transducer array 24 is inputted at T1, the operation for switching from the normal mode to the THI mode is carried out from the operation unit 16, before the next drive pulse Tx for transmitting the next ultrasonic wave is inputted at T2.

In this case, the controller 62 receives the control signal that commands mode switching from the controller 72 of the ultrasonic observing device 11, but the controller 62 keeps the switch 55 turned on between T1 and T2. Also, the variable capacitance capacitor Cᵥ is kept at C₁, and the angular frequency ω of the reference signal is kept at ω₁ in the quadrature detector 57, to drive the ultrasonic probe 12 in the normal mode. Accordingly, the reception signal Rx inputted to the reception circuit 56 has the fundamental frequency f₁ between T1 and T2.

Then, the controller 62 turns the switch 55 off at T2. The controller 62 also changes the variable capacitance capacitor Cᵥ to C₂, and changes the angular frequency ω of the reference signal to ω₂ in the quadrature detector 57, to drive the ultrasonic probe in the THI mode. Accordingly, after T2, the reception signal Rx inputted to the reception circuit 56 contains only the second harmonic component of the second reception signal outputted from the second ultrasonic transducer 42.

The ultrasonic diagnostic apparatus 10, as described above, is flexibly switchable between the normal mode and the THI mode, and the angular frequency ω of the reference signal used in the quadrature detector 57 is changed in accordance with the selected operation mode. Thus, even if the ultrasonic transducer array 24 is driven at the low voltage to reduce the power consumption, and the reception sensitivity is lowered with reduction in the transmission power, the quadrature detector 57 can enhance the frequency component that is necessary in each operation mode relative to the other frequency components by application of the quadrature detection processing. Especially in the THI mode, the second harmonic component is sensitively received, even if the ultrasonic transducer array 24 is driven at the lower voltage.

As described above, since the second ultrasonic transducer 42 is so designed as to mainly resonate with the second harmonic wave (frequency 2 f₁), the second ultrasonic transducer 42 can sensitively receive the second harmonic component without the resonant circuit 54. However, providing the resonant circuit 54 for the ultrasonic transducer 42 further enhances the reception sensitivity to the second harmonic component. Therefore, even if the ultrasonic transducer array 24 is driven at the low voltage, the ultrasonic image with the higher definition can be easily obtained in the THI mode.

In the ultrasonic diagnostic apparatus 10, the reception signals from the ultrasonic transducer array 24 are detected in the ultrasonic probe 12, and the reception signals are transmitted to the ultrasonic observing device 11 after serialization. Thus, it is possible to reduce the diameter of the cable 23 (or eliminate the cable 23) between the ultrasonic observing device 11 and the ultrasonic probe 12, and the ultrasonic probe 12 becomes easier to use.

In the above embodiment, the capacitance of the variable capacitance capacitor Cᵥ is set at C₁, when the ultrasonic diagnostic apparatus 10 is in the normal mode. In the case of observing a deep view (deep area) in the normal mode, the ultrasonic probe 12 is preferably driven with varying the capacitance of the variable capacitance capacitor Cᵥ as follows.

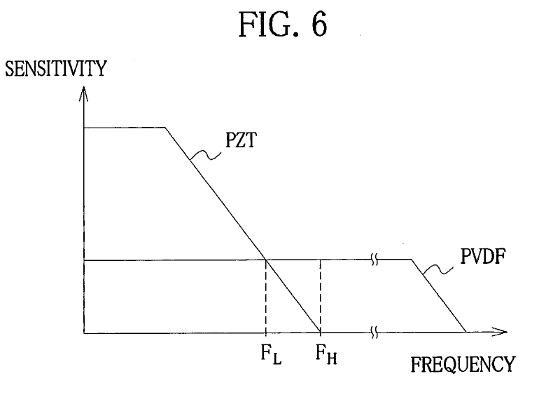

FIG. 6 shows the sensitivity characteristics of the first ultrasonic transducer 41 (PZT) and the second ultrasonic transducer 42 (PVDF). The sensitivity of the first ultrasonic transducer 41 is high at a low frequency band including the fundamental frequency f₁, and is gradually reduced with increase in the frequency above a certain frequency. On the other hand, the sensitivity of the second ultrasonic transducer 42 is approximately constant in a range of frequencies where the first ultrasonic transducer 41 is sensible, though the second ultrasonic transducer 42 is so designed as to resonate with the frequency 2 f₁ of the second harmonic wave. Accordingly, when f_{H} denotes the frequency at which the sensitivity of the first ultrasonic transducer 41 becomes almost zero, and f_{L} denotes the frequency at which the sensitivity of the first ultrasonic transducer 41 and the sensitivity of the second ultrasonic transducer 42 intersect in a graph, the second ultrasonic transducer 42 is more sensitive than the first ultrasonic transducer 41 to a signal in a frequency band between the frequencies f_{L} and f_{H}.

It is known that the ultrasonic wave attenuates in accordance with the magnitude of the frequency, concurrently with propagation distance. Especially inside the living body, it is known that the ultrasonic wave attenuates in proportion to the frequency. The echo from a deep point of the internal body part tends to lose a high frequency component, in comparison with the echo from a shallow point of the internal body part. Thus, even if the echo from the deep point has a center frequency of the fundamental frequency f₁ at the time of occurrence of the echo, the echo loses almost all signals in the frequency band (f_{L} to f_{H}), in which the sensitivities of the first and second ultrasonic transducers 41 and 42 are reversed, at the time of reception by the ultrasonic transducer array, 24. As a result, it'becomes impossible to produce the ultrasonic image with high definition with which tissue structure of the internal body part is observable.

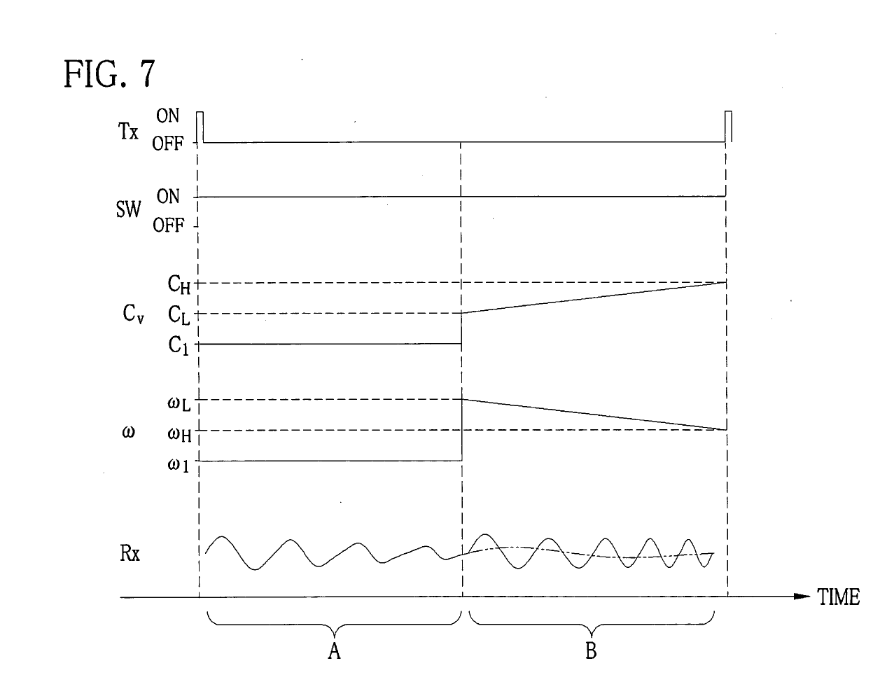

Thus, as shown in FIG. 7, when the echo from a shallow point A is received in the normal mode, the reception signal is obtained with setting the variable capacitance capacitor Cᵥ at C₁ and setting the angular frequency ω of the reference signal used in the quadrature detector 57 at ω₁, as described in the above embodiment. On the contrary, if the echo from a deep point B is received with setting the variable capacitance capacitor Cᵥ at C₁ and setting the angular frequency ω of the reference signal at ω₁, the reception signal Rx, as schematically shown by a chain double-dashed line, is useless for production of the ultrasonic image due to attenuation of high frequency components.

For this reason, in receiving the echo from the deep point B, the capacitance of the variable capacitance capacitor Cᵥ is proportionally increased from C_{L} to C_{H} with time. The capacitance C_{L} is determined by

$${f}_{L} = \frac{1}{2 π \sqrt{L \times {C}_{L}}} ,$$

and the capacitance C_{H} is determined by

$${f}_{H} = \frac{1}{2 π \sqrt{L \times {C}_{H}}} .$$

Similarly, in receiving the echo from the deep point B, the angular frequency ω of the reference signal used in the quadrature detector 57 is proportionally reduced from ω_{L} to ω_{H} with time. The angular frequency ω_{L} is determined by

$${ω}_{L} = \frac{{f}_{L}}{2 π} ,$$

and the angular frequency ω_{H} is determined by

$${ω}_{H} = \frac{{f}_{H}}{2 π} .$$

By setting the capacitance Cᵥ and the angular frequency ω as described above, the reception signal Rx produced from the echo from the deep point B majorly contains the second reception signal outputted from the second ultrasonic transducer 42.

As described above, in receiving the echo from the deep point B, if the attenuation of the high frequency component is small, setting the capacitance Cᵥ and the angular frequency ω as above within a frequency range (fundamental frequency range) originally receivable by the first ultrasonic transducer 41 is effective at obtaining the ultrasonic image with high definition that is available for diagnosis of the deep point B.

The depth of a border at which the capacitance Cᵥ is changed from C₁ to C_{L} (the angular frequency ω is changed from ω₁ to ω_{L}) is variable in accordance with input of the control signal from the operation unit 16. It is preferable that the depth at which the capacitance Cᵥ starts being changed from C₁ and the angular frequency ω starts being changed from ω₁ in the normal mode be automatically variable in accordance with the depth of a focus of the ultrasonic beam, the sound pressure of the ultrasonic beam, material properties of the body part to be imaged, and the like.

In the above embodiment, the ultrasonic probe 12 and the ultrasonic observing device 11 are connected via the cable 23. In the ultrasonic diagnostic apparatus 10, however, the reception signal is digitized and serialized in the ultrasonic probe 12, and then is transmitted to the ultrasonic observing device 11. Thus, a small-diameter cable for transmission of digital data is usable as the cable 23. It is preferable that the cable 23 used in the ultrasonic diagnostic apparatus 10 adhere to any standard of USB3.0, sATAgen2, and 10 GbaseT, for example. Use of such a small-diameter cable significantly facilitates handling of the ultrasonic probe 12.

The ultrasonic probe 12 and the ultrasonic observing device 11 are connected with the cable 23 in the above embodiment, but transmission and reception of data between the ultrasonic probe 12 and the ultrasonic observing device 11 may be carried out by wireless communication. In this case, the communication interfaces 61 and 73 are compliant with a wireless communication interface.

In the above embodiment, the first ultrasonic transducer 41 and the second ultrasonic transducer 42 are vertically stacked. However, the first ultrasonic transducers 41 and the second ultrasonic transducers 42 may be arranged in such a way that alternate arrangement in the AZ direction, parallel (two lines) arrangement, or the like.

The varicap is used as the variable capacitance capacitor Cᵥ in the above embodiment, but anything is available as the variable capacitance capacitor Cᵥ as long as the capacitance is variable.

In the above embodiment, the second ultrasonic transducer 42 is amenable to the reception of the second harmonic wave, but may be amenable to the third or more harmonic wave.

In the above embodiment, the first piezoelectric element 33 is made of PZT, and the second piezoelectric element 36 is made of PVDF. However, the first piezoelectric element 33 may be made of any piezoelectric material as long as the piezoelectric element can transmit and receive the ultrasonic wave of the fundamental frequency f₁, and the second piezoelectric element 36 may be made of any piezoelectric material as long as the piezoelectric material can receive the harmonic wave. However, if the first ultrasonic transducer 41 and the second ultrasonic transducer 42 are stacked as described above, it is preferable that the first piezoelectric element 33 for transmission and reception of the fundamental wave be made of the inorganic material such as PZT, and the second piezoelectric element 36 for reception of the harmonic wave be made of the organic material such as PVDF.

The present invention is applied to the portable ultrasonic diagnostic apparatus 10 in the above embodiment, but may be applicable to a stationary type of ultrasonic diagnostic apparatus.

In the above embodiment, the ultrasonic diagnostic apparatus 10 is switched from the normal mode to the THI mode in synchronization with input of the drive pulse Tx to the ultrasonic transducer array 24. However, the operation mode may be switched after output of the ultrasonic image of a frame, after the control signal for mode switching is inputted from the operation unit 16, for example. The same goes for switching from the THI mode to the normal mode.

Although the present invention has been fully described by the way of the preferred embodiment thereof with reference to the accompanying drawings, various changes and modifications will be apparent to those having skill in this field. Therefore, unless otherwise these changes and modifications depart from the scope of the present invention, they should be construed as included therein.

What is claimed is:

1. An ultrasonic diagnostic apparatus having an ultrasonic probe and an ultrasonic observing device, comprising:
(A) the ultrasonic probe, including:
an ultrasonic transducer array having a plurality of channels arranged in a line, each of the channels having a pair of a first ultrasonic transducer for transmitting and receiving an ultrasonic wave of a fundamental frequency and a second ultrasonic transducer for receiving a harmonic wave having a frequency of an integer multiple of the fundamental frequency;
a reception circuit for amplifying a first reception signal from the first ultrasonic transducer and a second reception signal from the second ultrasonic transducer, and applying analog-to-digital conversion to the first and second reception signals;
a detector for detecting an output signal from the reception circuit with use of a reference signal with a predetermined angular frequency;
a parallel-to-serial converter for converting an output signal from the detector into a serial signal;
a switching device for switching between a first mode and a second mode, in the first mode, the first reception signal from the first ultrasonic transducer and the second reception signal from the second ultrasonic transducer being added on a pair basis and inputted to the reception circuit, and in the second mode, only the second reception signal from the second ultrasonic transducer being inputted to the reception circuit; and
a controller for changing the angular frequency of the reference signal in accordance with a state of the switching device; and
(B) the ultrasonic observing device for producing an ultrasonic image from the serial signal transmitted from the ultrasonic probe.

2. The ultrasonic diagnostic apparatus according to claim 1, further comprising:
a resonant circuit having a variable resonant frequency disposed between the second ultrasonic transducer and the reception circuit, the controller determining the angular frequency in accordance with the resonant frequency.

3. The ultrasonic diagnostic apparatus according to claim 2, wherein the controller sets the resonant frequency at the fundamental frequency in the first mode, and set the resonant frequency at a frequency of the harmonic wave in the second mode.

4. The ultrasonic diagnostic apparatus according to claim 2, wherein in the first mode, the resonant frequency is changed in accordance with reception timing of the ultrasonic wave.

5. The ultrasonic diagnostic apparatus according to claim 2, wherein the resonant circuit includes an inductor and a variable capacitance capacitor connected in parallel, and the resonant frequency is adjusted by varying a capacitance of the variable capacitance capacitor.

6. The ultrasonic diagnostic apparatus according to claim 5, wherein the variable capacitance capacitor is a varicap.

7. The ultrasonic diagnostic apparatus according to claim 1, wherein
the first ultrasonic transducer has a piezoelectric element made of an inorganic material; and
the second ultrasonic transducer has a piezoelectric element made of an organic material.

8. The ultrasonic diagnostic apparatus according to claim 7, wherein the pair of the first ultrasonic transducer and the second ultrasonic transducer are stacked.

9. The ultrasonic diagnostic apparatus according to claim 1, wherein the ultrasonic probe and the ultrasonic observing device are portable.

10. The ultrasonic diagnostic apparatus according to claim 1, further comprising:
a cable for transmitting the serial signal from the ultrasonic probe to the ultrasonic observing device, the cable adhering to one of standards of USB3.0, sATAgen2, and 10 GbaseT.

11. The ultrasonic diagnostic apparatus according to claim 1, wherein the serial signal is transmitted by wireless communication from the ultrasonic probe to the ultrasonic observing device.
